(19) United States
(12) Patent Application Publication
(10) Pub. No.: US 2019/0008759 A1
(43) Pub. Date: Jan. 10, 2019

(54) SAFER AND MORE EFFECTIVE METHODS OF TRANSMUCOSAL DELIVERY FOR RAISING BLOOD PRESSURE AND STIMULATING THE BODY

(71) Applicant: DARREN RUBIN, LARGO, FL (US)

(72) Inventor: DARREN RUBIN, LARGO, FL (US)

(21) Appl. No.: 16/130,325

(22) Filed: Sep. 13, 2018

Related U.S. Application Data
(63) Continuation-in-part of application No. 15/818,056, filed on Nov. 20, 2017, now abandoned.
Application No. 15/818,056 is a continuation-in-part of application No. 14/323,561, filed on Jul. 3, 2014.

(51) Int. Cl. A61K 9/00; A61K 31/137

(52) U.S. Cl. CPC A61K 9/006; A61K 31/137; A61K 9/0056

(57) ABSTRACT

The present invention provides novel methods of mimicking epinephrine plasma pharmacokinetic parameters/plasma epinephrine levels of an at least one l-epinephrine injection in humans with an at least one dosage of a sublingual l-epinephrine formulation; and includes methods of maintaining constant elevated plasma epinephrine level(s) by the consecutive dosing of sublingual l-epinephrine. These methods are especially important when l-epinephrine injection is not available or not possible. The present invention allows small, l-epinephrine sublingual tablets to be carried by soldiers and others, such as in a remote location or battlefield, when emergency medical services are not readily available or accessible. The inventive methods can sustain life and restore proper blood perfusion when someone is having cardiopulmonary difficulty until medical help or transport can arrive.

### RELATED APPLICATION

The present application is a continuation-in-part of pending U.S. patent application Ser. No. 15/818,056 filed Nov. 20, 2017, which is a continuation-in-part of pending U.S. patent application Ser. No. 14/323,561 filed Jul. 3, 2014, the subject matter of which applications is incorporated herein by reference.

### FIELD OF THE INVENTION

The present invention provides novel methods of mimicking epinephrine plasma pharmacokinetic parameters/plasma epinephrine levels of an at least one l-epinephrine injection in humans with an at least one dosage of a sublingual l-epinephrine formulation; and includes methods of maintaining constant elevated plasma epinephrine level(s) by the consecutive dosing of sublingual l-epinephrine. These methods are especially important when l-epinephrine injection is not available or not possible. The present invention allows small, l-epinephrine sublingual tablets to be carried by soldiers and others, such as in a remote location or battlefield, when emergency medical services are not readily available or accessible. The inventive methods can sustain life and restore proper blood perfusion when someone is having cardiopulmonary difficulty until medical help or transport can arrive.

### BACKGROUND OF THE INVENTION

Adequate blood pressure provides the necessary perfusion of blood to tissues so that they receive oxygen and nutrients essential to their metabolic needs. A prolonged drop in blood pressure reduces blood flow and oxygen to these tissues. Eventually, this can lead to the state of circulatory shock; whole body circulatory failure by which tissues and organs lose perfusion, leading to irreparable, ischemic cellular injury, multiple organ failure, and death. Without immediate treatment, the chances of survival are greatly diminished, and cardiac arrest and respiratory arrest can ensue.

The main components of blood pressure include cardiac output, in terms of heart rate and stroke volume, which is related to heart size and strength of ventricular contraction; along with vascular resistance to flow, which is a function of peripheral vessel vasoconstriction.

Excessive peripheral vasodilation and vessel leakiness occur in conditions like systemic inflammatory response syndrome (SIRS), and in anaphylaxis and sepsis, which lead to anaphylactic shock and septic shock. These types of shock are associated with a decreased systemic vascular resistance and are distributive in nature; a subset of circulatory shock. With anaphylaxis, a severe allergic reaction, such as to a food or insect bite, triggers massive histamine release by mast cells, resulting in vasodilation. With sepsis, a blood infection, endotoxins released from blood borne pathogens, such as gram negative bacteria, lead to low perfusion by damaging endothelial cells of the vessels to release vasodilators, such as nitric oxide. Endotoxins also activate the complement pathway to release histamine from mast cells, another vasodilator. Endotoxins also activate macrophages and neutrophils of the immune system that release cytokines for a pro-inflammatory cascade that further damages endothelial cells, leading to increased vascular permeability or leakiness of the blood vessels that can decrease intravascular fluid volume. Both conditions are associated with a significant drop in blood pressure.

Hemodynamics can at least temporarily be restored by the emergency administration of Epinephrine Injection; the chemically synthesized drug product form of this endogenous catecholamine hormone, otherwise known as adrenaline, produced by the adrenal gland. Epinephrine acts on both alpha- and beta-adrenergic receptors. The mechanism of the rise in blood pressure is from epinephrine's three-fold actions of direct myocardial stimulation that increases the strength of ventricular contraction (positive inotropic action), an increased heart rate (positive chronotropic action), and peripheral vasoconstriction. Its action on alpha-adrenergic receptors is what lessens both the vasodilation and increased vascular permeability, which occur during anaphylaxis and sepsis.

Epinephrine Injection has a rapid onset and short duration of action. In a hospital setting, Epinephrine Injection is administered intravenously, e.g., by continuous intravenous infusion for treating hypotension associated with septic shock. But staying alive and getting to an emergency room, or having emergency medical services arrive at the scene, is not always possible in a timely manner. For individuals susceptible to allergic reactions, such as certain asthmatics and those with peanut allergy, autoinjectors of Epinephrine Injection are often prescribed and carried around for intramuscular (IM) or subcutaneous injection.

Epinephrine autoinjectors are not without issue. Firstly, they are large and bulky to carry around, especially since a second unit may be needed. So some patients may not always have the autoinjector with them. Then there are issues with storing them out of the heat (e.g., a hot glove compartment may degrade the product) and replacing them often (e.g., annually) due to a short shelf-life. Because of their high cost, some families forgo this expense altogether at great risk, while some use product well beyond its expiration date. Confusion about instructions of use with proper technique can also be an issue. If a patient triggers the device prematurely, the device becomes useless, and the dose does not get administered. Even when a patient has an epinephrine autoinjector, many are reluctant to use it because of needle fear. No doubt these autoinjectors hurt badly. The long needle is under great tension by a strong spring, and pushes outward with tremendous force. If an adult administers an autoinjector to a struggling child, cases of large cuts or gashes along the leg have been reported. And with the obesity epidemic, many experts believe autoinjector needles may not be long enough to provide intramuscular injection to all patients.

There is a tremendous need for a safer alternative, as well as, a more convenient and universal method of administering epinephrine to restore hemodynamics in an emergency situation, such as for anaphylaxis, sepsis, anaphylactic shock, septic shock, or even respiratory difficulty and cardiac difficulty; to sustain life and restore proper blood perfusion when someone is having cardiopulmonary difficulty until medical help or transport can arrive. If epinephrine was available in tablet form, many of the above issues and limitations of epinephrine autoinjectors would be eliminated. In tablet form, several doses of epinephrine can easily be carried and associated with a much longer shelf-life than autoinjectors, and fear of needles would not deter its use. The problem is that epinephrine is not conducive to oral administration. Epinephrine quickly degrades in the stomach/intestines and liver, and therefore, has poor oral bioavailability when ingested.

The term transmucosal drug delivery is drug delivery through a mucous membrane. Mucous membranes, or the mucosae, comprise the linings of bodily cavities and are of mostly endodermal origin and covered in epithelium, which are involved in absorption and secretion. Most absorption by the body takes place at the intestinal mucosa after oral, or enteral, administration of food and oral medications. Enteral means “through the intestines,” and may also include through the rectal mucosa by enema or suppository. But other mucosa are able to absorb medications, including the oral and buccal mucosa lining the mouth, the bronchial mucosa inside the lungs, and the nasal mucosa inside the nose. This invention relates to these non-intestinal mucosae, and preferably relates to non-intestinal mucosae residing inside the mouth, including sublingual, sublabial, buccal, palate, and gingiva sites inside a patient's mouth. Because the swallowing of epinephrine results in its rapid degradation in the stomach and metabolization in intestinal mucosa and the liver, the novelty of this invention includes methods which help avoid the swallowing of epinephrine when administered by transmucosal routes inside the mouth. These methods also apply to other hemodynamic agents to raise blood pressure.

So although the sublingual route represents a potential opportunity, sublingual administration of epinephrine powder or epinephrine orally disintegrating tablets or films can be swallowed accidentally, or saliva mixing with the disintegrated or dissolved product can be swallowed. This would result in loss of epinephrine due to the degradation of epinephrine associated with the oral route and first-pass metabolism by enzymes of the gastrointestinal tract and liver. The present invention provides methods of l-epinephrine sublingual administration and dosing for l-epinephrine sublingual tablets that have been proven to be effective in humans in the first of its kind clinical study. The present invention provides novel methods of non-intestinal transmucosal drug delivery of epinephrine, and or other adrenergic or vasoconstricting pharmaceutical agents (e.g., vasopressin and angiotensin II), that reduces swallowing of drug or saliva laden drug, which would otherwise reduce bioavailability and efficacy with inconsistent and suboptimal dosing. Only the present invention provides oral transmucosal delivery that ensures blood perfusion is restored in an emergency situation. The present invention guarantees that most of the epinephrine or other active pharmaceutical ingredient is transported to the circulation inside the tongue, mouth, or cheek to reach the body's systemic circulation. The present invention provides methods of elevating plasma epinephrine levels and or mimicking epinephrine plasma pharmacokinetic parameters/plasma epinephrine levels of an at least one injection of an injectable liquid l-epinephrine formulation, temporarily and or for an extended period of time, in a human with an at least one dosage of a sublingual l-epinephrine formulation. In this way, the present invention provides all of the advantages and none of the disadvantages inherent in prior methods and formulations. The present invention fulfills this great medical need in a safe, reliable, and pain-free method.

### BRIEF DESCRIPTION OF THE DRAWINGS

The present invention will be better understood and objects other than those set forth above will become apparent when consideration is given to the following detailed description thereof. Such description makes reference to the annexed drawings demonstrating preferred embodiments of the invention wherein:

FIG. 1 shows in linear scale, the mean baseline corrected epinephrine plasma concentrations (pg/mL) plotted against nominal time (hours) for human subjects dosed with ADRENATAB™ l-epinephrine sublingual tablets made with the Tartrate Salt form of epinephrine active ingredient compared to human subjects dosed with a 0.3 mg intramuscular epinephrine injection into the lateral part of the thigh.

FIG. 2 shows in logarithmic scale, the mean baseline corrected epinephrine plasma concentrations (pg/mL) plotted against nominal time (hours) for human subjects dosed with ADRENATAB l-epinephrine sublingual tablets made with the Tartrate Salt form of epinephrine active ingredient compared to human subjects dosed with a 0.3 mg intramuscular epinephrine injection into the lateral part of the thigh.

FIG. 3 shows in linear scale, the mean baseline corrected epinephrine plasma concentrations (pg/mL) plotted against nominal time (hours) for human subjects dosed with ADRENATAB l-epinephrine sublingual tablets made with the Base form of epinephrine active ingredient compared to human subjects dosed with a 0.3 mg intramuscular epinephrine injection into the lateral part of the thigh.

FIG. 4 shows in logarithmic scale, the mean baseline corrected epinephrine plasma concentrations (pg/mL) plotted against nominal time (hours) for human subjects dosed with ADRENATAB l-epinephrine sublingual tablets made with the Base form of epinephrine active ingredient compared to human subjects dosed with a 0.3 mg intramuscular epinephrine injection into the lateral part of the thigh.

### DETAILED DESCRIPTION OF THE INVENTION

The present invention provides methods of l-epinephrine sublingual administration and dosing for l-epinephrine sublingual tablets that have been proven to be effective in humans in the first of its kind clinical study. This is believed to be the first ever clinical trial conducted of l-epinephrine sublingual tablets; conducted under Good Clinical Practices (GCPs), Good Laboratory Practices (GLPs), and Good Manufacturing Practices (GMPs). Sublingual epinephrine tablets have never been studied in humans before, so there was no idea what to expect. Only non-human preclinical/animal studies were performed previously.

Animal studies of sublingual epinephrine tablets [see the rabbit studies of Rawas-Qalaji et al (J Allergy Clin Immunol 2006, 117:398-403), as referenced in U.S. Pat. No. 9,877,921 and US 2012/0322884] do not relate to real-life dosing in humans, not only because the sublingual tablets are artificially and mechanically held in position in these animals with laboratory equipment, but because even gentle handling or restraining of small animals greatly increases their plasma levels of catecholamines, and especially increases epinephrine plasma levels [see Buhler et al (J Physiol 1978 March; 276: 311-320)]. Therefore, preclinical animal studies of sublingual epinephrine in the known literature cannot and should not be relied upon for human comparison because any non-endogenous dosing can be greatly offset by large spikes in endogenous epinephrine levels released by the adrenal gland in these animals as part of their intrinsic adrenaline ‘fight-or-flight’ response. Other pharmacokinetic parameters would also differ between humans and small animals (e.g., rabbits) when comparing intramuscular injections, and intramuscular injections to sublingual administration, because of the vast size, muscular, and blood volume differences between humans and small animals. A human's mouth and sublingual absorptive area is much greater than that of a rabbit.

Previously, it was believed that the only way to ensure that sublingual epinephrine would be absorbed, rather than ingested, would be to use an adhesive, saliva impermeable barrier surrounding the sublingual epinephrine formulation on all other sides except the side where the active ingredient portion of the formulation was contacting the mucosal surface in the mouth. This would prevent epinephrine active ingredient from the dissolved tablet from being washed away and ingested.

The present invention provides methods of non-intestinal transmucosal drug delivery of sublingual l-epinephrine formulations that reduce the need for such saliva impermeable barriers surrounding the sublingual epinephrine formulation. In a primary embodiment of these methods, a sublingual l-epinephrine tablet (e.g., a 35 mg to 50 mg l-epinephrine sublingual tablet) is placed under a patient's tongue, such as immediately after removal of the tablet from a dispensing container. The patient is instructed to hold his/her tongue down over the tablet, and to keep his/her tongue and mouth still and not to swallow saliva for at least 5 minutes. The sublingual l-epinephrine tablet preferably disintegrates within the first 1 minute under the tongue. After 5 minutes, the patient is instructed to swallow once only the excessive saliva above the tongue, while continuing to hold the tongue down over the disintegrated tablet to protect it from mixing with saliva above the tongue and being swallowed. The disintegrated tablet is held under the tongue without swallowing any saliva for an additional 5 minutes. At approximately 10 minutes after the tablet had been placed in the mouth under the tongue, the patient is instructed to swallow all of his/her saliva and any remaining medication under the tongue, and or the patient can rinse his/her mouth out with a liquid (e.g., water) and spit it out. A second tablet, a third tablet, and so on, can be consecutively administered in this manner. Accordingly, consecutive doses should be spaced apart by at least 10 minutes. For example, consecutive doses are administered about every 10 minutes. For example, consecutive doses are administered about every 30 minutes. This invention, therefore, includes a method of maintaining a constant/near constant elevated plasma epinephrine level above 40 pg/mL for at least one half hour by the consecutive administration of at least two dosages of an at least one sublingual l-epinephrine formulation.

In other embodiments, consecutive doses are spaced apart by up to 60 minutes or more.

When more rapid epinephrine loading is necessary, semi-consecutive doses may overlap and be spaced apart by 5 minute increments. The first tablet may be held under the tongue for the full 10 minutes as described above, while the second tablet may be held under the tongue for only 5 minutes (minutes 5 through 10 starting from the first dosage; placed under the tongue after the above tongue saliva is swallowed), before swallowing everything and or rinsing the mouth out at 10 minutes from the first dose (5 minutes after the second dose is placed). This scenario may be repeated.

Alternatively, the dosing can be adjusted so that each tablet is held under the tongue for about 5 minutes without any saliva surrounding the tongue being swallowed before then. Then at about 5 minutes, everything can be swallowed and or the mouth rinsed out, before administering the next tablet; repeated about every 5 minutes.

In dire circumstances, more than one tablet may be co-administered simultaneously and held under the tongue. This may be necessary when someone is losing consciousness and must self-dose if no one else is around. However, this method poses greater cardiovascular risk.

In each method of administrating sublingual l-epinephrine tablets, it is preferred for the patient's vitals to be monitored. At the very least, another individual should keep check of the patient's breathing and pulse rate. If no one else is around, and if possible, the patient should check their own pulse rate. The term ‘patient’ is used, but the recipient of the sublingual l-epinephrine tablet can be anyone; e.g., soldier, policeman, fireman, first responder, student, outdoorsman/hiker, sailor, truck driver, and so on. Ideally, the patient or individual receiving the sublingual tablet should have his/her head positioned upright, although the sublingual tablet can be administered in a supine position, especially if self-administered.

The above methods of the preferred embodiment employ the use of the tongue to hold the sublingual l-epinephrine formulation down against the floor of the mouth, while the bottom of the tongue covers and surrounds the sublingual l-epinephrine formulation, to seal it off as much as possible from saliva located in the mouth above the tongue from reaching and mixing with the sublingual l-epinephrine formulation under the tongue, so that most or all of the l-epinephrine is absorbed sublingually rather than being ingested with saliva.

These methods of administration are unique because epinephrine has never been administered as a sublingual tablet to humans before. These methods of administration are also unique and nonobvious because other drugs, when in the form of a sublingual tablet, allow swallowing of medicated saliva as soon as the tablet disintegrates under the tongue; e.g., Intermezzo® (zolpidem tartrate) sublingual tablet for treatment of insomnia, Nitrostat® sublingual nitroglycerin tablets for treatment of angina pectoris, and Buprenorphine sublingual tablets for treatment of opioid dependence. There is no hold time for not swallowing medicated saliva with these other drug products. Other sublingual drug products cannot make the inventive steps of the Applicant's methods obvious.

The Applicant tested these inventive methods in the first of its kind human clinical study of l-epinephrine sublingual tablets. This clinical study has not been published and is not publicly available. This was a randomized, crossover, comparator controlled clinical study that measured the bioavailability and descriptive pharmacokinetic data of different strengths of two different l-epinephrine sublingual tablet formulations (one containing l-epinephrine base active pharmaceutical ingredient and one containing l-epinephrine tartrate salt active pharmaceutical ingredient), while recording changes in subject vital signs including blood pressure and heart rate, changes in electrocardiograms, all while looking for signs of mucosa irritation in the mouth. Safety of the tablets was also evaluated. The comparator was an intramuscular injection of 0.3 mg per 0.3 mL of l-epinephrine injection. The study was conducted under fasted conditions. The descriptive pharmacokinetic data or plasma pharmacokinetic parameters included: maximum observed plasma concentration, time to maximum observed plasma concentration, area under the plasma concentration versus time curve from time 0 to the last measurable concentration, area under the plasma concentration versus time curve, extrapolated to infinity, terminal elimination rate constant, terminal elimination half-life, apparent clearance, and apparent volume of distribution. Epinephrine, was analyzed both with and without baseline correction, as there is endogenous epinephrine present in the body, as well as, non-endogenous epinephrine introduced from the tablets. Comparisons were made between the treatment groups.

Some examples of the qualitative and quantitative composition, including percent weight by weight (% w/w), of each component in the l-epinephrine sublingual tablets that were prepared and underwent stability testing are provided in Table 1 and Table 2 below. Table 1 describes the formulations containing Epinephrine Base active pharmaceutical ingredient, while Table 2 describes formulations containing Epinephrine Bitartrate (Salt) active pharmaceutical ingredient. Shown are l-epinephrine sublingual tablet strengths of 2.5 mg, 5 mg, and 50 mg, respectively. Please note that nearly double the amount of Epinephrine Bitartrate (Salt) active pharmaceutical ingredient is required for epinephrine equivalency to Epinephrine Base active pharmaceutical ingredient, so that each 4.548 mg of Epinephrine Bitartrate, USP is equivalent to 2.5 mg of Epinephrine, USP Base; each 9.096 mg of Epinephrine Bitartrate, USP is equivalent to 5 mg of Epinephrine, USP Base; and each 90.960 mg of Epinephrine Bitartrate, USP is equivalent to 50 mg of Epinephrine, USP Base.

TABLE 1
Sublingual tablet formulations made with Epinephrine, USP (Base form)

| | | Quantity | | Quantity | | Quantity | | |
|---|---|---|---|---|---|---|---|---|
| | | per Unit | | per Unit | | per Unit | | |
| | | for | | for | | for | | |
| | | 2.5 mg | | 5 mg | | 50 mg | | Pharmaceutical |
| No. | Component | (mg) | % w/w | (mg) | % w/w | (mg) | % w/w | Function |
| 1 | Epinephrine, | 2.500 | 1.56 | 5.000 | 3.12 | 50.000 | 31.25 | Active Ingredient |
| | USP | | | | | | | |
| 2 | Mannitol, USP | 91.356 | 57.10 | 88.856 | 55.54 | 43.856 | 27.41 | Diluent |
| | (Mannogem EZ | | | | | | | |
| | Spray Dried) | | | | | | | |
| 3 | Microcrystalline | 56.000 | 35.00 | 56.000 | 35.00 | 56.000 | 35.00 | Diluent |
| | Cellulose, NF | | | | | | | |
| | (Avicel PH-102) | | | | | | | |
| 4 | Crospovidone | 8.000 | 5.00 | 8.000 | 5.00 | 8.000 | 5.00 | Disintegrating |
| | (Type-A), NF | | | | | | | Agent |
| | (Polyplasdone XL) | | | | | | | |
| 5 | Sucralose, NF | 0.544 | 0.34 | 0.544 | 0.34 | 0.544 | 0.34 | Sweetening Agent |
| 6 | Magnesium | 1.600 | 1.00 | 1.600 | 1.00 | 1.600 | 1.00 | Lubricant |
| | Stearate, NF | | | | | | | |

| | | | | | | |
|---|---|---|---|---|---|---|
| Total weight | 160.000 | 100.00 | 160.000 | 100.00 | 160.000 | 100.00 |

TABLE 2
Sublingual tablet formulations made with Epinephrine Bitartrate, USP (Salt form)

| | | Quantity | | Quantity | | Quantity | | |
|---|---|---|---|---|---|---|---|---|
| | | per Unit | | per Unit | | per Unit | | |
| | | for | | for | | for | | |
| | | 2.5 mg | | 5 mg | | 50 mg | | Pharmaceutical |
| No. | Component | (mg) | % w/w | (mg) | % w/w | (mg) | % w/w | Function |
| 1 | Epinephrine | 4.548 | 2.84 | 9.096 | 5.69 | 90.960 | 56.85 | Active Ingredient |
| | Bitartrate, USP | | | | | | | |
| 2 | Mannitol, USP | 89.308 | 55.82 | 84.760 | 52.98 | 2.896 | 1.81 | Diluent |
| | (Mannogem EZ | | | | | | | |
| | Spray Dried) | | | | | | | |
| 3 | Microcrystalline | 56.000 | 35.00 | 56.000 | 35.00 | 56.000 | 35.00 | Diluent |
| | Cellulose, NF | | | | | | | |
| | (Avicel PH-102) | | | | | | | |
| 4 | Crospovidone | 8.000 | 5.00 | 8.000 | 5.00 | 8.000 | 5.00 | Disintegrating |
| | (Type-A), NF | | | | | | | Agent |
| | (Polyplasdone XL) | | | | | | | |
| 5 | Sucralose, NF | 0.544 | 0.34 | 0.544 | 0.34 | 0.544 | 0.34 | Sweetening Agent |
| 6 | Magnesium | 1.600 | 1.00 | 1.600 | 1.00 | 1.600 | 1.00 | Lubricant |
| | Stearate, NF | | | | | | | |

| | | | | | | |
|---|---|---|---|---|---|---|
| Total weight | 160.000 | 100.00 | 160.000 | 100.0 | 160.000 | 100.00 |

The manufacturing of the above tablets included the steps of dispensing, sifting, blending, and compression/tableting. Briefly, these tablets were prepared by dispensing and sifting active ingredient and some quantities of inactive ingredients with a sieve to make a first premix blend. Other quantities of inactive ingredient were dispensed and sifted with a sieve to make a second premix blend. Magnesium Stearate and also mannitol were also sifted with a sieve. Several rounds of blending with a blender for up to 10 minutes were applied as the first sifted premix blend was added, followed by the second sifted premix blend, as well as, additional sifted mannitol and sifted magnesium. The final blend was compressed into tablets with a multi-station tablet press. Tablets were packed into bottles with desiccant and labeled. Initially, 100 count tablet bottles were prepared and placed in stability chambers, including with accelerated conditions at 40 degrees Celsius. The tablets were tested by analytical laboratory methods during development, at release, and during the stability testing program. These tablets were found to be very stable with good l-epinephrine purity profiles and low impurities. These tablets were named, and bottles labeled as, ADRENATAB.

The l-epinephrine active pharmaceutical ingredients used to make these tablets (ADRENATAB tablets), i.e., Epinephrine Base active pharmaceutical ingredient and Epinephrine Bitartrate Salt active pharmaceutical ingredient, come supplied from their manufacturer as a powder with particles in the micrometer range; i.e., are mostly microparticles of epinephrine. Because human bioavailability was found good with these tablets and methods of the Applicant, it makes other methods of epinephrine tablet manufacture and use nonobvious and unnecessary. By way of example, the patent references of Rawas-Qalaji et al U.S. Pat. No. 9,877,921 and US 2012/0322884 describe “Epinephrine nanoparticles, methods of fabrication thereof, and methods for use thereof for treatment of conditions responsive to epinephrine.” Rawas-Qalaji et al's methods of making epinephrine nanoparticles involve the use of combining epinephrine with solvents, such as isopropyl alcohol, to form a mixture, which is then subjected to high pressure and temperature in a reaction chamber. Any marginal increase in absorption, if any, by making these nanoparticles would be countered by the creation of additional impurities, and especially the impurity dextrorotary, d-epinephrine that forms as levorotary, l-epinephrine racemizes in solvent and with temperature and pressure. Rawas-Qalaji et al does not address these issues of epinephrine racemization and does not check chirality, and therefore, one would not rely on these references. Moreover, Rawas-Qalaji et al uses excipients, such as chitosan, which appear not to be pharmaceutically acceptable. Chitosan is not found in the FDA's inactive ingredient database for approved drug products. Chitosan is derived from shellfish origin, of which, individuals may be highly allergic to shellfish. So creating an epinephrine tablet with an ingredient that may cause severe allergic reaction, e.g., chitosan, is counterproductive and counterintuitive when the purpose of the tablet is to treat severe allergic reactions in allergy susceptible individuals. Therefore, the references of Rawas-Qalaji et al are nonobvious and flawed.

Pharmacokinetic results of the Applicant's first human clinical trial demonstrated, based on the point estimates observed for baseline corrected epinephrine, that the rate of absorption (Cmax) is lower for tablets having the Tartrate Salt form of epinephrine when compared to those having the Base form of epinephrine, while the extent of absorption (AUC 0-t) was similar. As mentioned, the l-epinephrine sublingual tablets used in this human clinical study were named, and bottles labeled as, ADRENATAB™. ADRENATAB l-epinephrine sublingual tablets are identified in the Figures as either ADRENATAB Base (tablets made with l-epinephrine base active ingredient) or ADRENATAB Tartrate Salt (tablets made with l-epinephrine bitartrate [salt]). In the study, blood samples were collected for analysis at 1 hour before and at a half hour before dosing; at time zero; and at 5 minutes, 10 minutes, 15 minutes, 20 minutes, 30 minutes, 40 minutes, 1 hour, 1.5 hours, 2 hours, 3 hours, and 6 hours after tablet dosing. Plasma epinephrine concentrations were measured by a bioanalytical facility using validated methods.

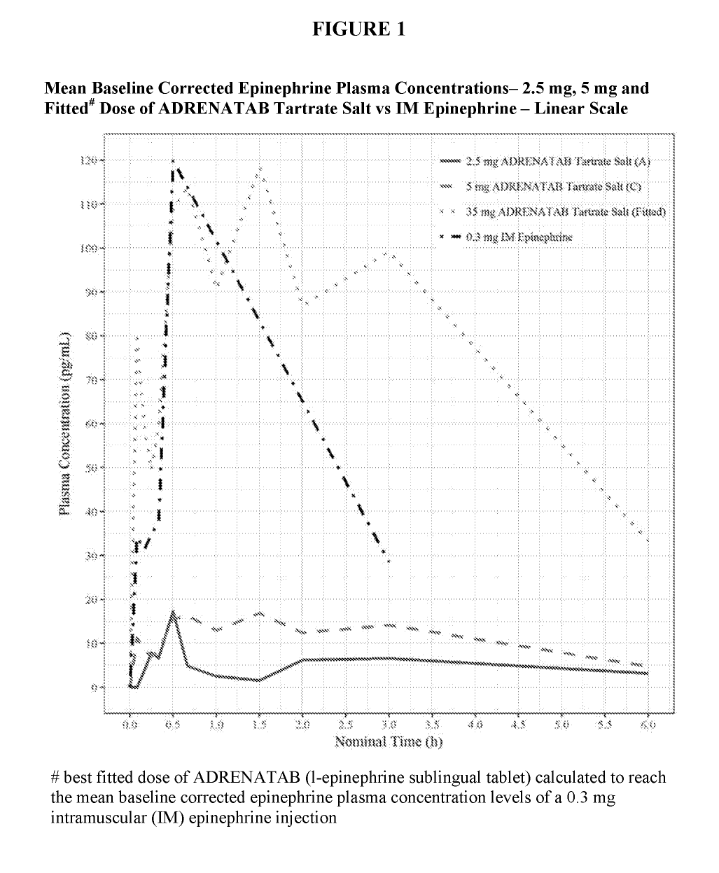

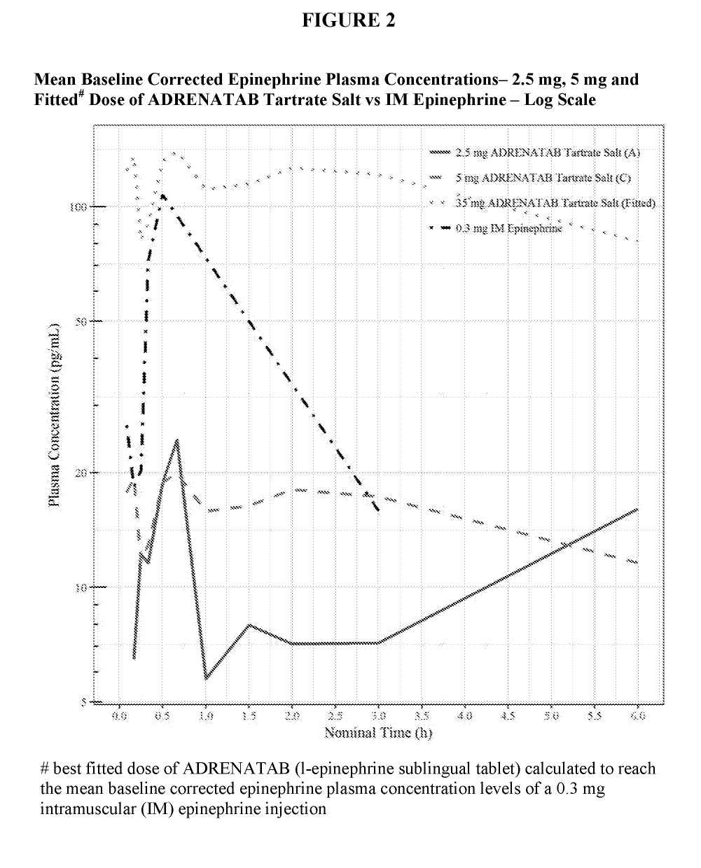

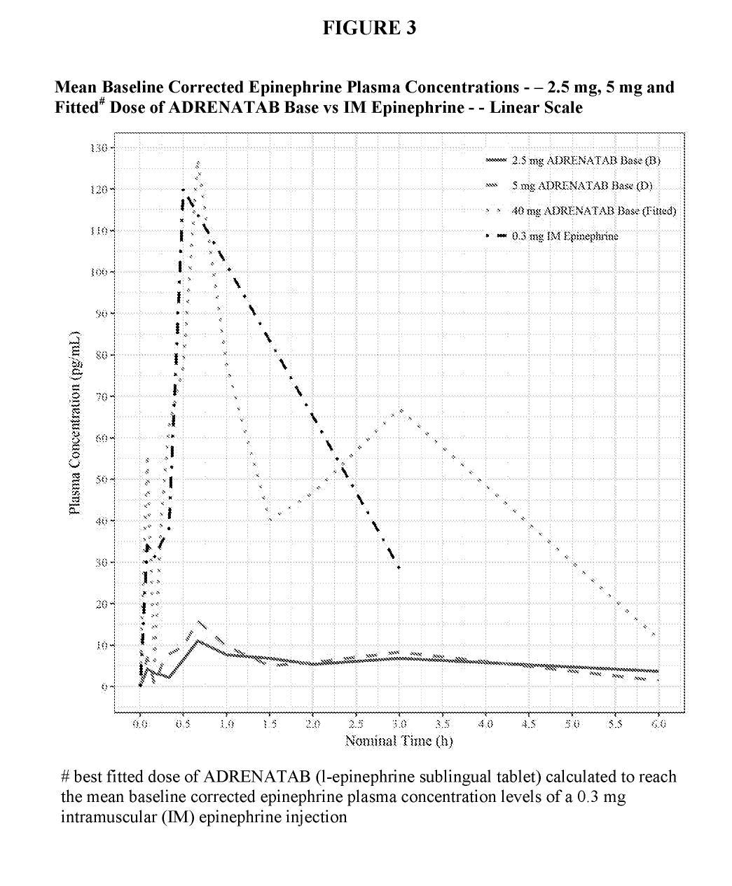

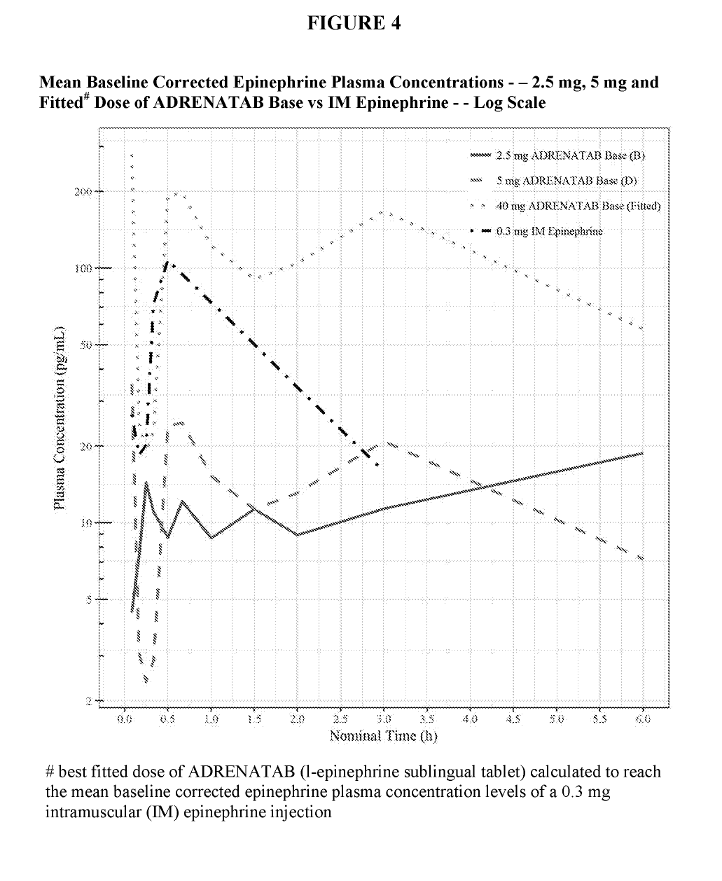

FIGS. 1-4 show mean baseline corrected epinephrine plasma concentrations (pg/mL) plotted against nominal time (hours) for subjects dosed with ADRENATAB l-epinephrine sublingual tablets compared to subjects dosed with a 0.3 mg intramuscular epinephrine injection into the lateral part of the thigh. Two of the smaller ADRENATAB tablet strengths of 2.5 mg and 5 mg are shown in these figures for reference to demonstrate that absorption took place even at these low dosages. Also shown is a calculated ADRENATAB tablet dose that best fits the mean baseline corrected epinephrine plasma concentrations following a 0.3 mg intramuscular (IM) epinephrine injection. For tablets made with Epinephrine Bitartrate, this fitted dose was calculated to be 35 mg, while for tablets made with Epinephrine Base, this fitted dose was calculated to be 40 mg. FIG. 1 and FIG. 2 show results of subjects dosed with the Epinephrine Bitartrate (Tartrate Salt) formulation of tablets, while FIG. 3 and FIG. 4 show results of subjects dosed with the Epinephrine Base formulation of tablets. FIG. 1 and FIG. 3 are shown in linear scale, while FIG. 2 and FIG. 4 are shown in logarithmic (Log) scale.

The Applicant's proposed methods of mimicking epinephrine plasma pharmacokinetic parameters/plasma epinephrine levels of an at least one injection of an at least one injectable liquid l-epinephrine formulation in humans with an at least one dosage of an at least one sublingual l-epinephrine formulation has been feasibly tested with the above clinical study conducted in humans. Pharmacokinetic data from this human clinical study demonstrates that elevated plasma epinephrine levels, including non-endogenous epinephrine, can be sustained for an extended period of time by the administration of l-epinephrine sublingual tablet(s).

The Applicant's methods help protect and prevent drug from mixing with saliva and being swallowed, thereby preventing both decreased bioavailability and loss of efficacy, and ensuring restoration of proper blood perfusion with the activity of the drug. The drug is preferably l-epinephrine and or other adrenergic or vasoconstricting pharmaceutical agents (e.g., norepinephrine, vasopressin, and angiotensin II). Some other future and or novel sublingual drugs may be administered using the Applicant's methods; such as sublingual insulin or an insulin analogue, insulin derivative, proinsulin, or drug that mimics insulin or insulin signaling, or a combination thereof.

In its simplest primary embodiment, the invention is a method of mimicking epinephrine plasma pharmacokinetic parameters/plasma epinephrine levels of an at least one injection of an at least one injectable liquid l-epinephrine formulation in a human with an at least one dosage of an at least one sublingual l-epinephrine formulation. The at least one injectable liquid l-epinephrine formulation is preferably at a concentration of 0.1 mg per mL to 1 mg per mL prior to any diluting. The at least one injectable liquid l-epinephrine formulation preferably contains 0.1 mg to 1 mg of l-epinephrine and or the at least one injectable liquid l-epinephrine formulation contains 0.1 mL to 10 mL of l-epinephrine prior to any diluting. The at least one injection of an at least one injectable liquid l-epinephrine formulation is selected from endotracheal injection, intracardiac injection, intramuscular injection, subcutaneous injection, subcutaneous infusion, intravenous injection, and intravenous infusion.

Such injectable liquid l-epinephrine formulations are administered to treat individuals and or patients who are experiencing: hypotension, such as may occur from drug or allergen hypersensitivity reactions, anaphylaxis, anaphylactic shock, sepsis, septic shock; respiratory distress, such as may occur by bronchospasm, asthma/asthma attack, respiratory infection; inadequate heart rhythm, such as may occur in cardiac arrest, arrhythmia, syncope/heart block, and those needing resuscitation; and mucosal congestion, such as may occur from allergy or sinus infection. In some instances, such injectable liquid l-epinephrine formulations can also be administered to prophylactically treat individuals and or patients who are experiencing the above. The Applicant's methods can achieve the above treatments and provide similar physiologic (pharmacodynamic) effects using sublingual l-epinephrine instead of an injection.

In most embodiments and methods, the at least one sublingual l-epinephrine formulation contains at least 10 mg of l-epinephrine and no more than 200 mg of l-epinephrine. In many embodiments and methods, the at least one sublingual l-epinephrine formulation contains at least 25 mg of l-epinephrine and no more than 70 mg of l-epinephrine.

In most preferred embodiments and methods, the at least one sublingual l-epinephrine formulation is in sublingual tablet form, and preferably an orally disintegrating sublingual tablet form.

In most preferred embodiments and methods, the at least one sublingual l-epinephrine formulation is in sublingual tablet form and contains at least one, and preferably at least two, pharmaceutically acceptable excipient ingredients.

In some preferred embodiments and methods, the at least one sublingual l-epinephrine formulation is in sublingual tablet form and contains at least one diluent excipient ingredient, at least one disintegrating agent excipient ingredient, at least one lubricant excipient ingredient, and optionally, at least one sweetening agent/taste masking agent excipient ingredient and or colorant.

In most preferred embodiments and methods, the at least one sublingual l-epinephrine formulation contains l-epinephrine active pharmaceutical ingredient that is not exposed to solvent and not exposed to drying processes during production/manufacturing of said sublingual l-epinephrine formulation. In other words, the l-epinephrine active pharmaceutical ingredient is not wetted and not exposed to solvents that would otherwise at least partially dissolve the l-epinephrine active pharmaceutical ingredient, which would require heat or drying to form a non-liquid formulation, and cause oxidation and racemization; i.e., degradation. Optionally, the l-epinephrine active pharmaceutical ingredient does not contain, or is not essentially comprised of, nanoparticles.

Importantly, in preferred embodiments and methods, the at least one sublingual l-epinephrine formulation contains less than 6% d-epinephrine impurity at release and contains less than 12% d-epinephrine impurity over a shelf-life of at least 24 months, and in most instances, longer.

In most preferred embodiments and methods, the at least one sublingual l-epinephrine formulation has a disintegration time no more than 2 minutes.

In many preferred embodiments and methods, the at least one sublingual l-epinephrine formulation has a disintegration time no more than 1 minute, and is preferably well under 1 minute.

A first preferred primary embodiment is a method of elevating and mimicking epinephrine plasma pharmacokinetic parameters/plasma epinephrine levels of an at least one injection of an at least one injectable liquid l-epinephrine formulation in a human with an at least one dosage of an at least one sublingual l-epinephrine formulation; this method further includes a first step of removing/dispensing the at least one dosage of the at least one sublingual l-epinephrine formulation from a dispensing container and placing the at least one dosage of the at least one sublingual l-epinephrine formulation under a human's tongue; this method further includes a second step of the human holding his/her tongue down over the at least one dosage of the at least one sublingual l-epinephrine formulation, while keeping the tongue and mouth still to protect the at least one dosage of the at least one sublingual l-epinephrine formulation from mixing with saliva above the tongue, and not swallowing any saliva for at least 5 minutes; this method further includes an optional third step of placing another at least one dosage of an at least one sublingual l-epinephrine formulation under the human's tongue.

A second preferred primary embodiment is a method of elevating and mimicking epinephrine plasma pharmacokinetic parameters/plasma epinephrine levels of an at least one injection of an at least one injectable liquid l-epinephrine formulation in a human with an at least one dosage of an at least one sublingual l-epinephrine formulation; this method further includes a first step of removing/dispensing the at least one dosage of the at least one sublingual l-epinephrine formulation from a dispensing container and placing the at least one dosage of the at least one sublingual l-epinephrine formulation under a human's tongue; this method further includes a second step of the human holding his/her tongue down over the at least one dosage of the at least one sublingual l-epinephrine formulation, while keeping the tongue and mouth still to protect the at least one dosage of the at least one sublingual l-epinephrine formulation from mixing with saliva above the tongue, and not swallowing any saliva for at least 5 minutes; this method further includes a third step of swallowing once (one time) any excessive saliva above the tongue, while continuing to hold the tongue down over the at least one dosage of the at least one sublingual l-epinephrine formulation; holding of said tongue down over said at least one dosage of the at least one sublingual l-epinephrine formulation continues for up to an additional 5 minutes; this method further includes a fourth step of mouth-clearing by swallowing all saliva and or mouth contents, and or rinsing the mouth out with a liquid and spitting; this method is optionally repeated as is needed or desired. This method was applied to dosing in the human clinical study mentioned above.

Some embodiments include a method of elevating and mimicking epinephrine plasma pharmacokinetic parameters/plasma epinephrine levels of an at least one injection of an at least one injectable liquid l-epinephrine formulation in a human with an at least one dosage of an at least one sublingual l-epinephrine formulation; this method further requiring at least two dosages of said at least one sublingual l-epinephrine formulation administered at least 5 minutes apart.

The invention includes a method of maintaining a constant/near constant elevated plasma epinephrine level above 40 pg/mL in humans for at least one half hour by the consecutive administration of at least two dosages of an at least one sublingual l-epinephrine formulation.

The invention includes a method of maintaining a constant/near constant raise in blood pressure by the administration of an at least one dosage of an at least one sublingual l-epinephrine formulation. The invention also includes a method of maintaining a constant/near constant raise in blood pressure for at least one half hour by the consecutive administration of at least two dosages of an at least one sublingual l-epinephrine formulation. The goal with this method is to normalize, or nearly normalize a hypotensive state to a normotensive blood pressure, or at the very least, increase blood pressure by a few mmHg to reduce the loss of perfusion as best it can.

The invention includes a method comprising the steps of repeat dosing of an at least one sublingual l-epinephrine formulation to elevate plasma epinephrine levels to maintain at least one of blood pressure, pulse, and breathing in a human patient having at least one of anaphylaxis, anaphylactic shock, sepsis, septic shock, respiratory difficulty, and cardiac difficulty; when an at least one injection of an injectable liquid l-epinephrine formulation is not available/not possible; at least until emergency medical services arrives to treat/transport the human patient; the human patient optionally comprising a soldier on a battlefield/remote location; this method optionally comprising the later step of administering the at least one injection of an injectable liquid l-epinephrine formulation if available/possible.

The Applicant's methods preferably ensures that most or more than half (more than 50%) of the active pharmaceutical ingredient of l-epinephrine from the pharmaceutical formulation of epinephrine is absorbed, without considerable racemization, and is prevented from being swallowed. The methods involve the tongue holding a formulation against a mucosa surface and blocking out all, nearly all, or most other saliva from surrounding areas/vicinities. Saliva from surrounding areas should not interact to a considerable extent. Alternative methods also couple these methods with a saliva impermeable barrier in the formulation and or associated with the formulation. In alternative embodiments, the barrier is chosen from physical structures including a non-absorbable removable layer of plastic or polymer material that provides a physical surface to which the pharmaceutical formulation of epinephrine is attached, at least temporarily, the physical structure further protecting/sheathing the pharmaceutical formulation of epinephrine from mixing with saliva outside of a zone/region where the pharmaceutical formulation of epinephrine is contacting and or secured/adhered to the mucous membrane.

Ideally, the sublingual l-epinephrine formulation contains no chitin, as chitin is derived from shellfish, which could induce an allergic reaction in susceptible individuals. Ideally, the sublingual l-epinephrine formulation contains no sulfites (e.g., no metabisulfite), which could induce an allergic reaction in susceptible individuals.

Preferred embodiments include no tartrate or bitartrate in the sublingual l-epinephrine formulation as epinephrine base appears better.

In some embodiments, the sublingual l-epinephrine formulation is associated with an at least one bioadhesive.

The invention is also a method of elevating plasma epinephrine levels (above normal baseline levels, or up to 40 pg/mL and or above 40 pg/mL) in a human with an at least one dosage of a sublingual l-epinephrine formulation. Primary embodiments include sublingual l-epinephrine orally disintegrating tablets. The invention optionally includes methods of elevating plasma epinephrine levels for an extended period of time, such as up to a half hour, or even beyond a half hour, by the administration of an at least one dosage of a sublingual l-epinephrine formulation. The invention is also a method of elevating plasma epinephrine levels (above normal baseline levels, or up to 40 pg/mL and or above 40 pg/mL) in a human with an at least one dosage of a sublingual l-epinephrine formulation containing at least one additional active pharmaceutical ingredient other than epinephrine, or an at least one dosage of a sublingual l-epinephrine formulation consecutively administered and or co-administered with an at least one formulation containing an active pharmaceutical ingredient other than epinephrine.

Conceivably, the invention is also a method of elevating plasma epinephrine levels, and or a method of mimicking epinephrine plasma pharmacokinetic parameters/plasma epinephrine levels of an at least one injection of an injectable liquid l-epinephrine formulation, temporarily and or for an extended period of time (e.g., a half hour), in a military soldier and or military pilot with an at least one dosage of a sublingual l-epinephrine formulation. The military can use the Applicant's methods and the Applicant's at least one dosage of a sublingual l-epinephrine formulation to not only treat a soldier experiencing at least one of hypotension, respiratory difficulty, and or cardiac difficulty, but also to enhance the physical and or mental training of soldiers, and also to stimulate soldiers during extreme combat situations.

Soldiers, like other individuals, may be sensitive to food allergens and insect bites which can trigger hypersensitivity reactions or anaphylaxis in susceptible individuals. The Applicant's invention negates the need for soldiers to carry around bulky and heat sensitive, liquid formulations of epinephrine, whether as an epinephrine autoinjector or epinephrine syringes, vials, or ampoules; and instead; carry a tiny container of one or more sublingual l-epinephrine tablets. However, unlike most individuals, soldiers are at greater risk of being exposed to many other chemical agents that may trigger anaphylaxis, whether it be from smoke inhalation of the burning of noxious materials or even from chemical warfare agents. The ATROPEN® is a prefilled auto-injector of Atropine Injection, an anticholinergic agent used to treat poisoning by susceptible organophosphorous nerve agents having cholinesterase activity as well as organophosphorous or carbamate insecticides. Atropine injection is believed to be part of an antidote kit prepared for the military. Likewise, sublingual l-epinephrine formulations, including tablets such as ADRENATAB, could be included in part of an antidote kit for soldiers. The Applicant's methods include the sequential dosing and or co-administration of an at least one sublingual l-epinephrine formulation and an at least one atropine and or hyoscyamine formulation, or other anticholinergic or antidote agent(s). These examples are not meant to be limiting. The atropine (or its levorotary isomer hyoscyamine) may be administered as a liquid injection and or as a solid oral formulation, and preferably as a sublingual formulation. In another preferred embodiment of the invention, a combination sublingual formulation is administered containing both l-epinephrine active ingredient and atropine (and or hyoscyamine) active ingredient as an antidote to nerve poisoning agent(s), or contain other anticholinergic or antidote agent(s). These examples are not meant to be limiting.

The Applicant's sublingual l-epinephrine formulation or dosage form and methods can also be used to enhance the physical and or mental training of combat soldiers and even combat vehicle drivers or fighter pilots by boosting blood perfusion throughout the brain and body with higher cardiac output, as well as, increasing brain metabolism and oxygen consumption by the brain. The invention may also simulate an ‘adrenaline rush’ during training that a soldier or pilot would otherwise experience out in the field. This may beforehand demonstrate the soldier's or pilot's ability to operate under the mental pressure and physical stress of combat or tactical missions.

Additionally, the Applicant's sublingual l-epinephrine formulation or dosage form and methods can also be used to stimulate soldiers and or pilots during extreme combat situations. Such stimulation is believed to reduce reaction time and reduce fear, and even temporarily reduce pain, such as from a wound (e.g., bullet wound). This would be useful if a soldier must charge several combatants in a high-risk situation or be in an intense dog fight with multiple combatants. With these methods, a soldier may impose maximum damage on the enemy. Perhaps these methods could provide at least some chance of survival in what would otherwise be a ‘suicide mission’. The Applicant's methods may also include the sequential dosing and or co-administration of an at least one sublingual l-epinephrine formulation and the administration of an at least one stimulant and or analgesic. The least one stimulant and or analgesic may be administered as liquid injection(s), topical/transdermal application(s), and or solid oral formulation(s), but preferably as sublingual formulation(s). In another preferred embodiment of the invention, a combination sublingual formulation is administered containing both l-epinephrine active ingredient and stimulant active ingredient (e.g., an amphetamine, e.g., a xanthine [caffeine or cocaine analogue]) and or analgesic active ingredient (e.g., a member of the opioid class, an opioid, such as morphine or fentanyl, or another opioid analogue, derivative, or a combination thereof) to enhance the ability of the soldier and or pilot during a combat situation. These examples are not meant to be limiting and other members of stimulant and or analgesic classes can be substituted or additionally included.

It is believed possible that the Applicant's sublingual l-epinephrine formulation or dosage form and methods can also be used to prevent, delay, or reduce the time that a combat pilot, or even astronaut, may lose consciousness (i.e., blackout) from high acceleration or gravitational force (e.g., more than a several-fold g-force or a maximum g-force).

Still further, it is believed the Applicant's sublingual l-epinephrine formulation or dosage form and methods can help revive a soldier after experiencing physical trauma or sedation.

Conceivably, the invention is also a method of elevating plasma epinephrine levels, and or a method of mimicking epinephrine plasma pharmacokinetic parameters/plasma epinephrine levels of an at least one injection of an injectable liquid l-epinephrine formulation, temporarily and or for an extended period of time (e.g., a half hour), in an athlete with an at least one dosage of a sublingual l-epinephrine formulation. These methods can help revive an athlete experiencing exercise induced hypotension, bronchospasm, asthmatic episode or cardiovascular collapse. Further, the Applicant's sublingual l-epinephrine formulation or dosage form and methods can also be used to enhance the physical and or mental training of athletes by boosting blood perfusion throughout the brain and body with higher cardiac output, as well as, increasing brain metabolism and oxygen consumption by the brain. The invention may also simulate an ‘adrenaline rush’ during athletic training that an athlete would otherwise experience out in the field during a game, race, or other athletic event or competition. Where permitted, when not considered ‘doping’, the Applicant's sublingual l-epinephrine formulation or dosage form and methods can also be used to stimulate (professional) athletes or Olympians during an event to enhance performance, reduce reaction time and reduce fear, and even temporarily reduce pain. The only issue is that if such methods were considered doping and banned, it would be very difficult to detect such l-epinephrine doping because one may not be able to test and distinguish elevated non-endogenous epinephrine levels from endogenous epinephrine. Moreover, because epinephrine is quickly inactivated by enzymes in the body, epinephrine levels would likely return to normal levels by the end of the event, and especially if methods of dosing/formulation were timed/adapted to do so. So detection of doping of l-epinephrine may be difficult to ascertain. Yet, it is interesting to imagine how l-epinephrine may enhance a sporting event such as football, wrestling, boxing, swimming, sprinting, etc. Epinephrine increasing blood flow to muscles and increasing blood glucose levels, along with cardiac output, should enhance athletic performance.

Further, the invention is also a method of elevating plasma epinephrine levels, and or a method of mimicking epinephrine plasma pharmacokinetic parameters/plasma epinephrine levels of an at least one injection of an injectable liquid l-epinephrine formulation, temporarily and or for an extended period of time (e.g., a half hour), for neurocognitive enhancement, especially if combined with a ‘smart drug,’ ‘smart supplement’, or other cognitive enhancer; a ‘nootropic’ agent. Such nootropic agents may include supplements such as ginseng and ginkgo biloba, and drugs such as racetams, e.g., piracetam and its derivatives. Nootropic agents can also include amphetamines (like Adderall® and Ritalin®), methylphenidate, modafinil, nicotine and their analogues, derivatives, and combinations thereof. These examples are not meant to be limiting. At least temporary neuroenhancement with the Applicant's methods may help in thinking of better strategies and solutions to problems in science, medicine, business, warfare, and help ‘think tanks’ and innovation as well. These methods can enhance blood flow and oxygen utilization by the brain and stimulate synaptic activity.

The above examples are meant to show that there can be additional properties and or synergies with the Applicant's methods and or of combining the Applicant's methods with one or more additional active ingredients.

The Applicant's methods even allow for a ‘cardiac stress test in a bottle’, as a method of simulating and or enhancing what is known as a cardiac diagnostic test/cardiopulmonary exercise test that usually requires response to an external stress (e.g., treadmill exercise) in a controlled environment. The Applicant's methods mimic the increase in plasma epinephrine levels that occurs with exercise, and or, mimics changes to a subject's vital signs that occur with exercise, by administering an at least one dosage of a sublingual l-epinephrine formulation according to the Applicant's invention.

In most embodiments, the pharmaceutical formulation of l-epinephrine is adapted to be immediately released for rapid transmucosal delivery. Still other embodiments of the invention can be envisioned, such as an extended release/sustained release sublingual l-epinephrine formulation or dosage form. A slow release or controlled release embodiment can contain up to 1,000 mg or more of epinephrine slowly released, such as to mimic intravenous infusion of epinephrine, such as for treating hypotension associated with septic shock. There can be other disease states, such as those associated with adrenal insufficiency, that may require slow, titrated epinephrine administration.

Alternative embodiments to the invention will now be described.

Other embodiments can include a dosage form that is a chewing gum, gummy candy, or hard candy/lozenge, or even as a sublingual breath spray. In still other embodiments, a patch (e.g., buccal patch) may be utilized, such as an epinephrine buccal patch. The tongue may be able to hold and cover a formulation to block saliva in the mouth from mixing with the formulation in other positions in the oral cavity. The at least one mucous membrane inside the patient's mouth is chosen from at least one mucosal site inside the patient's mouth, including, but not limited to, sublingual, sublabial, buccal, palate, and gingiva sites in the patient's mouth.

In some alternative embodiments, the methods may be at least somewhat similar to primary embodiments, however, the sublingual l-epinephrine formulation is optionally surrounded by a saliva impermeable physical barrier or excipient formulation on all sides except for the one side that contacts the sublingual mucosa where absorption takes place. This saliva impermeable physical barrier further protects the l-epinephrine active ingredient from interacting with saliva beyond the site of attachment. This physical structure or barrier, in some embodiments, can also help adhere the pharmaceutical formulation to the oral mucosa site and help keep it dry from saliva. In some of these alternative embodiments, this protective layer or physical structure, such as a film, membrane, or gel, dissolves to release the pharmaceutical active ingredient for delivery, and or dissolves after the active pharmaceutical ingredient has been delivered transmucosally. The timing of such dissolving of this protective layer or physical structure can be modified to achieve the desired dosing effect, and as such, the size, thickness, and composition of said protective layer or physical structure is chosen as such. In some embodiments, this protective layer or physical structure is bioabsorbable or degradable. In yet other embodiments, this protective layer or physical structure is non-absorbable, such as a plastic polymer, and instead must be physically removed from the oral cavity following transmucosal drug delivery, or must be swallowed and excreted by the patient following transmucosal drug delivery inside the oral cavity. When a non-absorbable protective layer or physical structure is adhered to or around the target mucosa, a warm liquid, such as water or alcohol may be required for its removal, along with rubbing the protective layer or physical structure with a finger, brush, utensil, washcloth, or another object to loosen the adhesive. In other embodiments, the adhesion can be negated by a change in pH, exposure to air, or other biologically safe solvents. In some embodiments, this protective layer or physical structure blocks at least some of the tasting of bad/bitter tasting pharmaceutical ingredients, and as such, can also prevent additional saliva secretion from taking place when an otherwise bad/bitter taste is experienced.

These methods provide a more consistent and reproducible dosage, with greater bioavailability and less metabolic/enzymatic degradation over prior methods of transmucosal delivery in the oral cavity. These methods are further associated with at least one of greater systemic bioavailability, lower dilution, lower side effects, or a combination thereof.

The pharmaceutical formulation of the invention includes at least one dosage form chosen from the class of dosage forms including, but not limited to, a liquid, liquid droplets, a gel, a paste, a film, a biofilm, a tape, a wafer, a membrane, a sheath, a disc, beads, a tablet, a caplet, lozenges, semi-solids, glues, gums, or a combination thereof. Capsules are also possible, such as capsules containing l-epinephrine active ingredient powder. The pharmaceutical formulation further includes at least one pharmaceutically acceptable excipient selected from pharmaceutical excipients including, but not limited to, carriers, solvents, solubilizing excipients, taste masking excipients, sweeteners, lipids, liposomes, adherents, bioadhesives, glycoproteins, proteins, carbohydrates, starches, waxes, polymers, charged/ionic excipients, non-charged/neutral excipients, nanoexcipients/nanoparticles, microencapsulating excipients, chelating excipients, excipients that dissolve over time, biocompatible excipients, non-absorbable excipients, and bioabsorbable excipients. The at least one pharmaceutically acceptable excipient is further chosen for the desired speed of transmucosal delivery, whether desired for immediate release and delivery, or slow release and sustained transmucosal delivery. The at least one pharmaceutically acceptable excipient is likewise chosen to aid in the passive diffusion and/or active transport of the at least one pharmaceutical active ingredient.

This invention also consists of at least two dosages of a pharmaceutical formulation. These two dosages are preferably two different dosage amounts. These at least two different dosages, in some embodiments, preferably have different releasing characteristics, for example, with layers or emulsions of excipients chosen for timed release or differential release. These at least two different dosages, in other embodiments, preferably have different size/area profiles and/or different concentrations per square unit of area in contact with a mucosal surface. Furthermore, each different dosage or dosage amount can be specific to a certain mucosal site in the oral cavity so that different dosages can be administered to different mucosae in the oral cavity. This method can allow the sites in the mouth to be rotated, so that the same mucosa site is not being dosed over and over again, so as to avoid local complications, such as localized toxicity or reduced transport at that site during a treatment regimen. Because transport efficiency and the vasculature may differ between different mucosae in the oral cavity, e.g., the palate versus buccal sites, dosages can be calibrated for these various mucosae. This method of dosage and mucosa site rotation can maintain efficient drug transport over a treatment regimen, whether formulated for active transport or passive diffusion, and whether formulated for immediate release or sustained transmucosal delivery.

In some embodiments, a collective dose is formed of smaller dosage aliquots comprised of one or more dosages from one or more different applicators. Combinations of different dosage amounts and different applicators provide different options to patients to ensure that certain mucosa are not being over treated with drug so as to reduce local side effects and to provide optimum bioavailability. These different applicator combinations can be rotated to provide the same collective (total) dose, without treating the same mucosa sites consecutively.

Another alternative embodiment of the invention includes a method of non-intestinal transmucosal delivery of a pharmaceutical formulation of l-epinephrine, including an active pharmaceutical ingredient of l-epinephrine and at least one pharmaceutically acceptable excipient, the method avoiding at least most first-pass metabolism by the liver by preventing saliva from mixing with this active pharmaceutical ingredient of l-epinephrine and being ingested; the method including administration of this pharmaceutical formulation of l-epinephrine inside a patient's mouth for transmucosal delivery across an at least one mucous membrane inside the patient's mouth. This method further includes adhering the pharmaceutical formulation of l-epinephrine to the at least one mucous membrane with a bioadhesive for transmucosal delivery. In some embodiments the bioadhesive is a mucoadhesive. This method still further includes sheathing the pharmaceutical formulation of l-epinephrine with a barrier to protect from mixing with saliva outside of a zone/region where the pharmaceutical formulation of l-epinephrine is adhered to the at least one mucous membrane, so that more than half (more than 50%) of the active pharmaceutical ingredient of l-epinephrine from the pharmaceutical formulation of l-epinephrine is prevented from being swallowed. The barrier comprises a hydrophobic layer, hydrophobic surface, physical barrier, or combination thereof at least partially surrounding the pharmaceutical formulation of l-epinephrine. This method is further associated with at least one of greater systemic bioavailability, lower dilution, lower side effects, or a combination thereof.

Further embodiments of the invention also include placing and adhering the pharmaceutical formulation to mucosa inside the oral cavity. The invention in tertiary embodiments also includes an applicator to aid in the placement of the pharmaceutical formulation inside the mouth and preferably aids in adhering the formulation to the target mucosa site. In some embodiments, the applicator is used to administer a single dosage of said pharmaceutical formulation. In other embodiments, the applicator is used to administer multiple dosages of one or more pharmaceutical formulations. In some of these embodiments, the applicator houses and dispenses multiple dosages of one or more pharmaceutical formulations of the invention with the bioadhesive.

In some other embodiments, l-epinephrine is accompanied with or replaced by an at least one other member of the class of adrenergic or vasoconstricting pharmaceutical agents. Examples of other adrenergic or vasoconstricting pharmaceutical agents of this class include norepinephrine, vasopressin, and angiotensin II). In other embodiments, l-epinephrine is accompanied by an at least one other pharmaceutical agent in the same pharmaceutical formulation or in a separate pharmaceutical formulation. In some other embodiments, l-epinephrine is accompanied by an at least one other pharmaceutical agent selected from the class of antihistamines, e.g., diphenhydramine, either in the same pharmaceutical formulation or in a separate pharmaceutical formulation.

Embodiments of this invention can include various numbers of excipient ingredients and/or percent weight/weight of these excipient ingredients. Excipient ingredients are selected from the classes of excipients including, but not limited to, antiadherents, binders, coatings, nanoparticles, chelators, buffering agents, acid reacting excipients, alkaline reacting excipients, disintegrants, fillers, diluents, colors/colorants, lubricants, glidants, preservatives, sorbents, flavors, sweeteners, carriers, solvents, surfactants, bioadhesives, mucoadhesives, and any mixtures and combinations thereof; and can include lipids, liposomes, glycoproteins, proteins, carbohydrates, saccharides, starches, waxes, and polymers.

An at least one bioadhesive excipient is an element and or an additional element to some methods, which includes adhering the pharmaceutical formulation of at least one active pharmaceutical ingredient, which in preferred embodiments is l-epinephrine, to at least one oral mucous membrane with the at least one bioadhesive for transmucosal delivery of said at least one active pharmaceutical ingredient. The active pharmaceutical ingredient (e.g., l-epinephrine) portion of the drug product may be secured or adhered directly or indirectly to the oral mucosal membrane. Generally, the active pharmaceutical ingredient (e.g., l-epinephrine) portion of the drug product is secured in contacting abutment to the oral mucosal membrane by surrounding bioadhesive of the drug product or physical structure; an indirect adhesion of the active pharmaceutical ingredient. In some instances, however, some bioadhesive may exist in the active pharmaceutical ingredient-containing formulation, allowing direct adhesion of the active portion of the drug product in contact with the oral mucosal membrane. In some instances, the bioadhesive may be at least somewhat permeable to the active pharmaceutical ingredient. So the language, “said method further including adhering said pharmaceutical formulation of active ingredient (e.g., l-epinephrine) to said at least one mucous membrane with a bioadhesive for said transmucosal delivery” accounts for both direct adhesion and or indirect adhesion of the pharmaceutical formulation of active ingredient and at least one mucous membrane.

Some alternative methods can also include sheathing the pharmaceutical formulation of the at least one active ingredient (e.g., l-epinephrine) with a barrier to protect from mixing with saliva outside of a zone/region where the pharmaceutical formulation of the at least one active ingredient is adhered to the at least one oral mucous membrane, to prevent the swallowing of medicated saliva. Therefore, in these alternative embodiments, the barrier at least partially comprises or is structurally and or functionally associated with the at least one bioadhesive excipient.

In many alternative embodiments, the bioadhesive excipient will be a bioadhesive polymer, which by definition is a synthetic or natural polymer which binds, sticks, or adheres to biological target tissue, namely in this case, mucosal membranes, at least temporarily. Mucosal membranes line externally exposed passageways of the body; such as the mouth, nose, respiratory tract, gastrointestinal tract, and vagina; with mucus which helps protect and keep moist these surfaces. Most embodiments of this invention are applied to the oral mucosal membranes, although, application to other mucosal membranes can be envisioned. Mucus contains water, mineral salts, and the glycoprotein known as mucin. The sublingual gland under the tongue produces mucin. The bioadhesive can stick to these moist, often mucousy, epithelial tissue surfaces. In some embodiments the bioadhesive is a mucoadhesive, in which case, interaction and or adhesion with mucus takes place.

For bioadhesion to occur, prolonged contact between the bioadhesive polymer(s) and the mucosal must be had for binding of bioadhesive polymers to the mucin/epithelial surface. Generally, the bioadhesive polymer(s) will wet and swell over the mucosal site, such as from interaction with moisture or saliva on the mucosal surface. It is believed that bioadhesive polymer chains will interdiffuse and intermingle with the mucosal membrane, and in some instances penetrating the mucus, and forming bonds with the entangled glycoprotein chains of the mucosa thereof. Stickiness may be associated with swelling. Different forces maintain the adhesion, whether cohesive, through van der Waals force, hydrogen bonding, ionic or chemical bonding, or a combination thereof.

The properties of the bioadhesion can be controlled via the selection of the bioadhesive materials, such as selecting bioadhesive polymers with the desired concentration, flexibility, hydrophilicity, swelling, and surface tension, and size/molecular weight of the bioadhesive polymer chains. The bioadhesive or bioadhesive formulation can be solid at first, like a tablet or lozenge, a powder, or be semi-solid, like a gel, membrane, or film. In some embodiments, the bioadhesive formulation can be a liquid or viscous liquid which forms a gel or solid upon interaction with saliva. The bioadhesive or bioadhesive formulation is therefore selected from the class consisting of powders, solids, tablets, lozenges, patches, semi-solids, gels, films, tapes, viscous liquids and liquids, bioadhesives that form a gel or solid, or any combination thereof. The bioadhesive is selected to have interaction at the proper pH of the oral mucosal environment. The bioadhesive is or becomes sticky after interacting with or absorbing fluid.

In some alternative embodiments, an at least one bioadhesive excipient is selected from the class of natural bioadhesive polymers and or the class of synthetic bioadhesive polymers, or analogue, derivative, or combination thereof. Some bioadhesives can be thought of as biocompatible glues, gums, pastes, or gels. Examples of members of the class of natural bioadhesive polymers include acacia gum, alginic acid, gelatin, guar gum, pectin, sodium alginate, sodium hyaluronate, and tragacanth. Examples of members of the class of semi-synthetic and synthetic bioadhesive polymers include carbomers and other polyacrylics, carboxymethyl cellulose, hydroxypropyl cellulose, hydroxypropyl methylcellulose, polycarbophil, polyethylene glycol, polyvinyl alcohol, polyvinylpyrrolidone, and silicone polymers. An example of a bioadhesive paste includes gelatin, pectin, xanthan gum, polyethylene glycol, carboxymethylcellulose, mineral oil, and optionally, one or more parabens. This is different from an adhesive oral paste formulation used for many decades containing gelatin, pectin, and carboxymethylcellulose. An example of a bioadhesive film applied as a gel includes boric acid, hydroxypropylcellulose, propylene glycol, purified water, salicylic acid, denatured alcohol, and tannic acid. Lactose, acacia, and magnesium stearate can be used as a bioadhesive for a buccal tablet, and can be by example, in a proportion, e.g., 10 mg, 30 mg, and 10 mg; although need not be in whole number ratios.

While the bioadhesive(s) holds the at least one active ingredient or pharmaceutical formulation of active ingredient(s) in place on the oral mucosa according to this alternative method, the active ingredient will absorbed transmucosally either by passive diffusion, facilitated passive diffusion, active transport, pinocytosis, or a combination thereof. The sheathing of the at least one active ingredient or pharmaceutical formulation of active ingredient(s) protects against the swallowing of medicated saliva and from interfering with transmucosal absorption/delivery.

The bioadhesive layer or barrier is preferably hydrophilic on the side facing the mucosal surface, to absorb moisture from the mucus layer and become sticky; while preferably being hydrophobic and moisture repellent on the opposite side, so as to prevent other saliva from entering the zone/region of the drug product and to prevent binding to mucosa surfaces on both sides. There can be some instances where the bioadhesive layer or barrier is preferably hydrophilic on both sides, including when adhering to at least two mucosal surfaces simultaneously is desired. These instances will generally include adherence to the corner of the gingiva, where it interfaces with the cheek or floor of the mouth. It can also be desired to have the bioadhesive stick to the bottom tongue surface and the floor of the mouth simultaneously. When at least two mucosal surfaces are bound simultaneously by the same drug product dosage, it can be desirable for transmucosal delivery to also occur at the at least two mucosal surfaces simultaneously, for enhanced efficiency or quickness of transmucosal delivery. Either way, adherence to at least two mucosal surfaces simultaneously can greatly increase the total adherence, total adherence surface area, ensuring that the drug product is secure and will not be swallowed during transmucosal delivery. A larger drug dose and or extended release drug dose can be had with stronger/longer bioadhesion times. This is important since treating hypotension from anaphylaxis may require multiple doses of epinephrine, and extended release may minimize the number of repeat doses.

It is desirable for the bioadhesive of the method to rapidly adhere to the mucosa, adhere adequately or strongly, and without interfering with drug release and transmucosal delivery. It is best for the bioadhesive to not be influenced by the food and drink recently taken by the patient; minimal food effects. If the patient's mouth is dry or wet, it is desirable for the bioadhesive to bind well to the mucosa in either case.

It is also desirable for the bioadhesive to work well with drug penetration enhancers, such as having a liposomal formulation (e.g., liposomal epinephrine), or have some drug penetration properties itself for transmucosal delivery, such as containing dimethyl sulfoxide (DMSO) solvent. The bioadhesive would facilitate contact of the active ingredient (e.g., epinephrine) with the underlying surface under the mucus layer. Again, the drug product may be secured and or adhered directly and or indirectly to the oral mucosal membrane by bioadhesive.

In embodiments with a bioadhesive, transmucosal delivery of the at least one active pharmaceutical ingredient including l-epinephrine, finishes well before bioadhesion stops, such as well before mucin turnover occurs. In these embodiments, the bioadhesive can be manually removed from the patient's oral mucosa after a period of time, for example after at least 1 minute, and preferably 2 minutes. In other embodiments, the bioadhesive is a natural or safe bioadhesive that can dissolved or be chewed and swallowed or spit out after the drug has been delivered.

A saliva barrier that is a bioadhesive/bioadhesive formulation or a saliva barrier with a bioadhesive/bioadhesive formulation is adjacent to or at least partially surrounding the pharmaceutical formulations of epinephrine for transmucosal delivery in some alternative embodiments. The barrier preferably includes polymers. The bioadhesive preferably includes gums. The epinephrine formulation can therefore be associated with hydroxypropylcellulose, hydroxypropyl methylcellulose, polyvinylpyrrolidine and or propylene glycol, and Acacia or Xanthan gum as barrier/bioadhesive. Other embodiments can include pregelatinised starch. The amounts of these barrier/bioadhesive materials/excipients can vary depending on the strength and longevity of the desired bioadhesive function, and are not meant to be limiting. Some embodiments can also include menthol so the user can feel the dosage on the mucosal surface working.

Other embodiments can be envisioned whereby epinephrine or a liquid formulation thereof is spray dried onto beads or another surface, such as the bottom surface of a tablet or physical barrier. In such embodiments, epinephrine or its formulation becomes a coat or coating layer to a surface or physical barrier to this invention. The inventive methods according to this invention can be carried out in further ways, and these examples are not meant to be limiting.

These methods can include at least two dosages of the pharmaceutical formulation of epinephrine. The at least two dosages of the pharmaceutical formulation of epinephrine can be in two different dosage amounts/concentrations of the pharmaceutical formulation of epinephrine.

When the method includes at least two different dosages/dosage amounts/dosage concentrations of the pharmaceutical formulation of epinephrine, each of the at least two different dosages/dosage amounts/dosage concentrations of the pharmaceutical formulation of epinephrine can be specifically calibrated for transmucosal delivery to a different mucous membrane inside an oral cavity, chosen from mucosal surfaces inside a patient's mouth, including, but not limited to, sublingual, sublabial, buccal, palate, and gingiva surfaces in the patient's mouth, so that dosage administration of the pharmaceutical formulation of epinephrine can be rotated among different mucosal surfaces inside the patient's mouth to prevent local complications of drug delivery to any one such the mucosal surface during a treatment regimen, the local complications of drug delivery including localized side effects or reduced diffusion/transport.

In some situations, such as when the mouth has been damaged, methods also include rectal administration in a human with an at least one dosage of a sublingual (or transmucosal) l-epinephrine formulation. One can envision veterinary uses in large animals, such as dogs and horses.

The transmucosal delivery is by passive diffusion across a mucous membrane and or by active transport across a mucous membrane. In some alternative embodiments, an electrical charge can be applied to the formulation via the applicator device to drive the transmucosal delivery.

Interestingly, these methods can provide temporary enhanced speed, physical strength, or physical endurance in the patient or user. This can have interesting applications for the military, federal agents, law enforcement agents, first responders, and even athletes. Superhuman ability may be needed in emergency situations or to accomplish above normal feats.

The Applicant's methods include holding and or protecting an at least one oral transmucosal formulation with the tongue over a minute. The Applicant's methods include holding and or protecting an at least one sublingual formulation under the tongue over a minute. The Applicant's methods include holding and or protecting an at least one sublingual tablet formulation under the tongue over a minute. The Applicant's methods include holding and or protecting an at least one l-epinephrine sublingual tablet formulation under the tongue over a minute, and preferably over 2 minutes or more. The Applicant's methods include consecutive dosing of holding and or protecting an at least one sublingual formulation under the tongue. The Applicant's methods include consecutive dosing of holding and or protecting an at least one l-epinephrine sublingual tablet under the tongue.

Other variations and embodiments of the invention described herein will now be apparent to those of skill in the art without departing from the disclosure of the invention or the coverage of the claims to follow.

What is claimed is:

1. A method of mimicking epinephrine plasma pharmacokinetic parameters/plasma epinephrine levels of an at least one injection of an at least one injectable liquid l-epinephrine formulation in a human with an at least one dosage of an at least one sublingual l-epinephrine formulation.

2. The method of claim 1 whereby said at least one injectable liquid l-epinephrine formulation is at a concentration of 0.1 mg per mL to 1 mg per mL prior to any diluting.

3. The method of claim 1 whereby said at least one injectable liquid l-epinephrine formulation contains 0.1 mg to 1 mg of l-epinephrine.

4. The method of claim 1 whereby said at least one injectable liquid l-epinephrine formulation contains 0.1 mL to 10 mL of l-epinephrine prior to any diluting.

5. The method of claim 1 whereby said at least one injection of said at least one injectable liquid l-epinephrine formulation is selected from endotracheal injection, intracardiac injection, intramuscular injection, subcutaneous injection, subcutaneous infusion, intravenous injection, and intravenous infusion.

6. The method of claim 1 whereby said at least one sublingual l-epinephrine formulation contains at least 10 mg of l-epinephrine and no more than 200 mg of l-epinephrine.

7. The method of claim 1 whereby said at least one sublingual l-epinephrine formulation contains at least 25 mg of l-epinephrine and no more than 70 mg of l-epinephrine.

8. The method of claim 1 whereby said at least one sublingual l-epinephrine formulation is in sublingual tablet form.

9. The method of claim 1 whereby said at least one sublingual l-epinephrine formulation is surrounded by a saliva impermeable barrier on all sides except for one side that contacts a sublingual mucosa where absorption takes place.

10. The method of claim 1 whereby said at least one sublingual l-epinephrine formulation contains at least two pharmaceutically acceptable excipient ingredients.

11. The method of claim 1 whereby said at least one sublingual l-epinephrine formulation is in sublingual tablet form and contains at least one diluent excipient ingredient, at least one disintegrating agent excipient ingredient, at least one lubricant excipient ingredient, and optionally, at least one sweetening agent/taste masking agent excipient ingredient.

12. The method of claim 1 whereby said at least one sublingual l-epinephrine formulation contains l-epinephrine active pharmaceutical ingredient that is not exposed to solvent and not exposed to drying processes during production/manufacturing of said at least one sublingual l-epinephrine formulation, and optionally said l-epinephrine active pharmaceutical ingredient is not essentially nanoparticles.

13. The method of claim 1 whereby said at least one sublingual l-epinephrine formulation has a disintegration time no more than 1 minute.

14. The method of claim 1 whereby said at least one sublingual l-epinephrine formulation contains less than 6% d-epinephrine impurity at release and contains less than 12% d-epinephrine impurity over a shelf-life of at least 24 months.

15. The method of claim 1 further including a first step of removing/dispensing said at least one dosage of said at least one sublingual l-epinephrine formulation from a dispensing container and placing said at least one dosage of said at least one sublingual l-epinephrine formulation under a human's tongue; said method further including a second step of said human holding said tongue down over said at least one dosage of said at least one sublingual l-epinephrine formulation, while keeping said tongue and mouth still to protect said at least one dosage of said at least one sublingual l-epinephrine formulation from mixing with saliva above said tongue, and not swallowing any saliva for at least 5 minutes; said method further including an optional third step of placing another said at least one dosage of said at least one sublingual l-epinephrine formulation under said human's tongue.

16. The method of claim 1 further including a first step of removing/dispensing said at least one dosage of said at least one sublingual l-epinephrine formulation from a dispensing container and placing said at least one dosage of said at least one sublingual l-epinephrine formulation under a human's tongue; said method further including a second step of said human holding said tongue down over said at least one dosage of said at least one sublingual l-epinephrine formulation, while keeping said tongue and mouth still to protect said at least one dosage of said at least one sublingual l-epinephrine formulation from mixing with saliva above said tongue, and not swallowing any saliva for at least 5 minutes; said method further including a third step of swallowing once any excessive saliva above the tongue, while continuing to hold said tongue down over said at least one dosage of said at least one sublingual l-epinephrine formulation; holding of said tongue down over said at least one dosage of said at least one sublingual l-epinephrine formulation continues for up to an additional 5 minutes; said method further including a fourth step of mouth-clearing by swallowing all saliva/contents and or mouth rinsing with a liquid and spitting out; said method optionally being repeated.

17. The method of claim 1 further requiring at least two dosages of said at least one sublingual l-epinephrine formulation administered at least 5 minutes apart.

18. The method of claim 1 whereby said at least one sublingual l-epinephrine formulation contains no chitin, contains no sulfites, and optionally contains no tartrate/bitartrate.

19. A method of maintaining a constant/near constant elevated plasma epinephrine level above 40 pg/mL in humans for at least one half hour by the consecutive administration of at least two dosages of an at least one sublingual l-epinephrine formulation.

20. A method comprising the steps of repeat dosing of an at least one sublingual l-epinephrine formulation to elevate plasma epinephrine levels to maintain at least one of blood pressure, pulse, and breathing in a human patient having at least one of anaphylaxis, anaphylactic shock, sepsis, septic shock, respiratory difficulty, and cardiac difficulty; when an at least one injection of an injectable liquid l-epinephrine formulation is not available/not possible; at least until emergency medical services arrives to treat/transport said human patient; said human patient optionally comprising a soldier on a battlefield/remote location; said method optionally comprising the later step of administering said at least one injection of an injectable liquid l-epinephrine formulation if available/possible.
